(19) United States
(12) Patent Application Publication
(10) Pub. No.: US 2005/0165082 A1
(43) Pub. Date: Jul. 28, 2005

(54) Methods of treating vasomotor symptoms

(75) Inventors: Liza Leventhal, Lawrenceville, NJ (US); Robert H. Ring, West Windsor, NJ (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 11/013,019

(22) Filed: Dec. 15, 2004

Related U.S. Application Data
(60) Provisional application No. 60/529,930, filed on Dec. 16, 2003.

(51) Int. Cl. A61K 31/405

(52) U.S. Cl. 514/414

(57) ABSTRACT

The present invention relates to methods of treating at least one vasomotor symptom such as hot flush, caused by, inter alia, thermoregulatory dysfunction, in a subject in need thereof by administering to the subject a compound or composition of compounds that modulate the V_{1b} receptor.

### CROSS REFERENCE TO RELATED APPLICATIONS

This application claims the benefit of U.S. Application No. 60/529,930 filed Dec. 16, 2003, the entire disclosure of which is herein incorporated by reference.

### FIELD OF THE INVENTION

The present invention relates to methods of treating vasomotor symptoms, in particular, using compounds and compositions of compounds that modulate the biological activity of the V_{1b} receptor for maintaining normal thermoregulatory homeostasis.

### BACKGROUND OF THE INVENTION

Vasomotor symptoms (VMS), referred to as hot flushes and night sweats, are the most common symptoms associated with menopause, occurring in 60% to 80% of all women following natural or surgically-induced menopause. It is well recognized that VMS are caused by fluctuations of sex steroid levels and can be disruptive and disabling in both males and females. The patient experiences a hot flash as a sudden feeling of heat that spreads quickly from the face to the chest and back and then over the rest of the body. It is usually accompanied by outbreaks of profuse sweating. A hot flush can last up to thirty minutes and vary in their frequency from several times a week to multiple occurrences per day. It may sometimes occur several times an hour, and it often occurs at night. Hot flushes and outbreaks of sweats occurring during the night can cause sleep deprivation. Psychological and emotional symptoms observed, such as nervousness, fatigue, irritability, insomnia, depression, memory loss, headache, anxiety, nervousness or inability to concentrate are considered to be caused by the sleep deprivation following hot flush and night sweats (Kramer et al., In: Murphy et al., 3^{rd} Int'l Symposium on Recent Advances in Urological Cancer Diagnosis and Treatment-Proceedings, Paris, France: SCI: 3-7 (1992)).

Hot flushes may be even more severe in women treated for breast cancer for several reasons: 1) many survivors of breast cancer are given tamoxifen, the most prevalent side effect of which is hot flush, 2) many women treated for breast cancer undergo premature menopause from chemotherapy, 3) women with a history of breast cancer have generally been denied estrogen therapy because of concerns about potential recurrence of breast cancer (Loprinzi, C. L., et al., Lancet, 2000, 356(9247): 2059-2063).

Men also experience hot flushes following steroid hormone (androgen) withdrawal. This is true in cases of age-associated androgen decline (Katovich, et al., Proceedings of the Society for Experimental Biology & Medicine, 1990, 193(2): 129-35) as well as in extreme cases of hormone deprivation associated with treatments for prostate cancer (Berendsen, et al., European Journal of Pharmacology, 2001, 419(1): 47-54. As many as one-third of these patients will experience persistent and frequent symptoms severe enough to cause significant discomfort and inconvenience.

The precise mechanism of these symptoms is unknown but generally is thought to represent disturbances to normal homeostatic mechanisms controlling thermoregulation and vasomotor activity (Kronenberg et al., “Thermoregulatory Physiology of Menopausal Hot Flashes: A Review,” Can. J. Physiol. Pharmacol., 1987, 65: 1312-1324).

To date, the most effective therapies for VMS are hormone-based treatments, including estrogens and/or some progestins. For example, the menopausal stage of life is associated with a wide range of other acute symptoms as described above and these symptoms are generally estrogen responsive. The fact that estrogen treatment (e.g. estrogen replacement therapy) relieves the symptoms establishes the link between these symptoms and an estrogen deficiency.

Although hormonal treatments are very effective at alleviating VMS, they are not appropriate for all women (Berendsen, Maturitas, 2000. 36(3): p. 155-164, Fink et al., Nature, 1996. 383(6598): p. 306). In particular, hormone replacement therapy is usually not recommended for women or men with or at risk for hormonally sensitive cancers (e.g. breast or prostate cancer). Thus, non-steroidal therapies (e.g. fluoxetine, paroxetine [SRIs] and clonidine) are being evaluated clinically. WO9944601 discloses a method for decreasing hot flushes in a human female by administering fluoxetine. Other options have been studied for the treatment of hot flashes, including steroids, alpha-adrenergic agonists, and beta-blockers, with varying degree of success (Waldinger et al., Maturitas, 2000. 36(3): p. 165-168).

Arginine-vasopressin (AVP) is a hormone known for a number of central and peripheral functions in mammal. Among them are water and solute excretion by kidney, blood pressure control, vascular smooth muscle cell and uterine contraction, platelet aggregation, liver glycogenolysis and neoglucogenesis, adrenocorticotropin (ACTH) release by the adenohypophysis and clotting factor release (Drug News Perspect. 1999 12 279-292).

AVP stimulates several types of receptors: V₁ (V₁ₐ, V_{1b}), V₂. These receptors are located in the liver, the vessels (coronary, renal and cerebral), the platelets, the kidneys, the uterus, the adrenal glands, the pancreas, the central nervous system and the pituitary gland. AVP thus exerts cardiovascular, hepatic, pancreatic, antidiuretic and platelet-aggregating effects as well as effects on the central and peripheral nervous system, and on the uterine sphere (J. Lab. Clin. Med., 1989, 114,(6), 617-632 and Pharmacol. Rev., 1991, 43(1), 73-108.

V_{1b} receptors have been cloned in rats, man and mice (Y. De Keyser, FEBS Letters, 1994, 356, 215-220; T. Sugimoto et al., J. Biol. Chem. 1994, 269(43), 27088-27092; M. Saito et al., Biochem. Biophys. Res. Commun., 1995, 212(3), 751-757; S. J. Lolait et al., Neurobiology, 1996, 92, 6783-6787; M. A. Ventura et al., Journal of Molecular Endocrinology, 1999, 22, 251-260). Moreover, the human V_{1b} receptor has been cloned and characterized (J. Biol. Chem. 1994 269, 27089-27092; Am. J. Physiol. Endocrinol. Metab. 2003 285 E566-E576).

Further, there is evidence suggesting that V_{1b} receptor may be associated with regulation of stress response (Current Drug Targets—CNS Neurological Disorders, 2003 2 191-200).

WO 93/15051, EP-A-0,636,608. EP-A-0,636,609, WO 95/18105, WO 97/15556 and WO 98/25901 have described substituted 1,3-dihydro-2H-indol-2-ones as arginine-vasopressin receptor ligands and/or oxytocin receptor ligands. Further, a selective V_{1b} receptor antagonist (SSR149415) has been developed that exhibits anxiolytic and anti-depressant-like activity (Progress in Brain Research 2002 139 197-210; PNAS 2002 99 6370-6375; Stress 2003 6 199-206, J. Pharmacol. Exp. Ther. 2002 300: 1122-1130).

However, there is no reported link between a V_{1b} receptor activity and the treatment of vasomotor stability or normalization of thermoregulatory homeostasis.

Given the multifaceted nature of thermoregulation, multiple therapies and approaches can be developed to target vasomotor instability. The present invention provides methods of treating vasomotor symptoms by modulating the biological activity of V_{1b} receptor.

### SUMMARY OF THE INVENTION

The present invention directs to compounds and compositions containing compounds to modulate the activity of the V_{1b} receptor for the prevention and treatment of, inter alia, vasomotor symptoms (VMS) caused by, for example, thermoregulatory dysfunctions, such as those experienced by pre-, peri- and post menopausal females and naturally, chemically or surgically andropausal males. In some aspects, the present invention relates to the use of compounds and compositions which modulate the activity of the V_{1b} receptor. In other aspects, the present invention provides the use of a compound or composition of compounds which modulate the activity of the V_{1b} receptor for the manufacture of a medicament for the treatment of conditions associated with vasomotor instability such as hot flush. Such compounds or composition of compounds include pharmaceutical compositions and products containing V_{1b} receptor antagonist activity and a pharmaceutically acceptable carrier.

In one embodiment, the present invention directed to methods for treating at least one vasomotor symptom in a subject in need thereof, comprising the step of:
administering to said subject an effect amount of a composition, comprising:
at least one compound that modulate the biological activity of V_{1b} receptor or pharmaceutically acceptable salt thereof, preferably the compound is V_{1b} receptor antagonist.

In other embodiments, the invention is directed to methods for identifying an agent for treating at least one vasomotor symptom in a subject, comprising the steps of:
growing a cell or tissue sample in the presence and absence of a test agent, wherein said cell or tissue sample expresses a V_{1b} receptor;
determining the biological activity of a V_{1b} agonist at said V_{1b} receptor in the presence and the absence of said agent; and
identifying said agent that antagonizes or reduces said biological activity of said V_{1b} agonist.

In another embodiment, the invention is directed to methods for screening to identify an agent for treating at least one vasomotor symptom in a subject, comprising the step of:
determining a binding affinity of said agent to a V_{1b} receptor.
   The methods optionally comprise the further step of determining the ability of said agent to displace binding of vasopressin to said V_{1b} receptor.

In further embodiments, the invention is directed to methods for screening to identify an agent for treating at least one vasomotor symptom in a subject, comprising the step of:
determining a binding affinity of said agent to cells or membranes expressing a V_{1b} receptor.

In yet other embodiments, the invention is directed to pharmaceutical compositions for treating at least one vasomotor symptom in a subject, comprising:
a. at least one V_{1b} receptor antagonist or a pharmaceutically acceptable salt thereof; and
b. at least one pharmaceutically acceptable carrier.

### BRIEF DESCRIPTION OF THE DRAWINGS

The invention can be more fully understood from the following detailed description and the accompanying drawings that form a part of this application.

FIG. 1: Panel A: Changes in TST (Δ° C., Mean) over time in the morphine-dependent rat model depict significant differences between groups. Panel B: At maximal flush (15 minutes post-naloxone; Δ° C., Mean±SEM), V_{1b} receptor antagonism (SSR149415) significantly and dose-dependently abated the naloxone-induced flush at 30 mg/kg, but had no effect at 3.0 mg/kg. (* indicates p<0.01 compared to vehicle control). Panel C: Changes in TST (Δ° C., Mean) over time in a telemetry model of thermoregulatory dysfunction. Administration of the V_{1b} receptor antagonist SSR149415 (30 mg/kg) resulted in a significant decrease in ΔTST relative to vehicle treatment. (* indicates p<0.05 compared to vehicle control) (referred to in Example 1).

FIG. 2: At maximal flush (15 minutes post-naloxone; Δ° C., Mean±SEM), both the V₁ₐ receptor antagonist SR49059 and V₂ receptor antagonist VPA-985 failed to abate the naloxone-induced flush. (* indicates p<0.05 compared to vehicle control) (referred to in Example 2).

### DETAILED DESCRIPTION OF THE INVENTION

The present invention directs to compounds and compositions containing compounds to modulate the activity of the V_{1b} receptor for the prevention and treatment of, inter alia, vasomotor symptoms (VMS) caused by, for example, thermoregulatory dysfunctions, such as those experienced by pre-, per- and post menopausal females and naturally, chemically or surgically andropausal males. In some aspects, the present invention relates to the use of compounds and compositions which modulate the activity of the V_{1b} receptor. In other aspects, the present invention provides the use of a compound or composition of compounds which modulate the activity of the V_{1b} receptor for the manufacture of a medicament for the treatment of conditions associated with vasomotor instability such as hot flush. Such compounds or composition of compounds include pharmaceutical compositions and products containing V_{1b} receptor antagonist activity and a pharmaceutically acceptable carrier.

It is believed that the present invention described presents a substantial breakthrough in the field of treatment, alleviation, inhibition, and/or prevention of vasomotor instability and/or dysfunction and hence treatment of thermoregulatory disorders.

In one embodiment, the present invention directed to methods for treating at least one vasomotor symptom in a subject in need thereof, comprising the step of:
administering to said subject an effect amount of a composition, comprising:
at least one compound that modulate the biological activity of V_{1b} receptor or pharmaceutically acceptable salt thereof, preferably the compound is V_{1b} receptor antagonist.

In another embodiment, the compound that modulates the activity of V_{1b} receptor is a compound which inhibits the biological activity of V_{1b} receptor, such as V_{1b} receptor antagonist.

In another embodiment, it was discovered that using V_{1b} receptor antagonists, substituted 1,3-dihydro-2h-indol-2-ones, result in an improved treatment to maintain normal thermoregulatory homeostasis.

Examples of V_{1b} receptor antagonist include, but are not limited to:
(2S,4R)-1-[5-chloro-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl) sulphonyl]-3-(2-methoxyphenyl)-2-oxo-2,3-dihydro-1H-indol-3-yl]-4-methoxy-N,N-dimethyl-2-pyrrolidine-carboxamide;
(2S,4R)-1-[5-chloro-3-(2-chlorophenyl)-1-[(2,4-dimethoxyphenyl)sulphonyl]-2-oxo-2,3-dihydro-1H-indol-3-yl]-4-hydroxy-N,N-dimethyl-2-pyrrolidinecarboxamide;
(2S,4R)-1-[5-chloro-3-(2-chlorophenyl)-1-[(2,4-dimethoxyphenyl)sulphonyl]-6-methoxy-2-oxo-2,3-dihydro-1H-indol-3-yl]-4-methoxy-N,N-dimethyl-2-pyrrolidine-carboxamide;
(2S,4R)-1-[5-chloro-1-[(3,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide;
methyl(2S,4R)-1-[5-chloro-3-(2-methoxyphenyl)-1-[(3,4-dimethoxyphenyl)sulphonyl]-2-oxo-2,3-dihydro-1H-indol-3-yl]-4-hydroxy-2-pyrrolidinecarboxylate;
(2S,4R)-1-[5-methyl-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl)-sulphonyl]-3-(2-methoxyphenyl)-2-oxo-2,3-dihydro-1H-indol-3-yl]-2-(azetidin-1-ylcarbonyl)-4-hydroxy-pyrrolidinecarboxamide;
(2S,4R)-1-[5-trifluoromethoxy-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidinecarboxamide;
(2S,4R)-1-[5-chloro-1-[(2,4-dimethoxyphenyl)sulphonyl]-3-(2-methoxyphenyl)-6-methyl-2-oxo-2,3-dihydro-1H-indol-3-yl]-4-hydroxy-N,N-dimethyl-2-pyrrolidinecarboxamide;
(2S,4R)-1-[3-(2-chlorophenyl)-1-[(2,4-dimethoxyphenyl)sulphonyl]-5,6-dimethyl-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl) sulphonyl]-3-(2,3-dimethoxyphenyl)-2-oxo-2,3-dihydro-1H-indol-3-yl]-4-methoxy-N,N-dimethyl-2-pyrrolidine-carboxamide;
(2S,4R)-1-[5-chloro-1-[(2,4-dimethoxyphenyl)sulphonyl]-3-(2-methoxyphenyl)-6-trifluoromethyl-2-oxo-2,3-dihydro-1H-indol-3-yl]-4-methoxy-N,N-dimethyl-2-pyrrolidinecarboxamide;
(2S,4R)-1-[6-chloro-1-[(2,4-dimethoxyphenyl)sulphonyl]-3-(2-methoxyphenyl)-5-methyl-2-oxo-2,3-dihydro-1H-indol-3-yl]-4-methoxy-N,N-dimethyl-2-pyrrolidinecarboxamide;
(2S,4R)-1-[5-chloro-1-[(2,4-dimethoxyphenyl) sulphonyl]-3-(2-methoxyphenyl)-2-oxo-2,3-dihydro-1H-indol-3-yl]-4-ethoxy-N,N-dimethyl-2-pyrrolidine-carboxamide;
(2S,4R)-1-[5-chloro-1-[(2,4-dimethoxyphenyl)sulphonyl]-3-(2,3-dimethoxyphenyl)-2-oxo-2,3-dihydro-1H-indol-3-yl]-4-hydroxy-N,N-dimethyl-2-pyrrolidine-carboxamide;
(2S,4R)-1-[5,6-dichloro-3-(2-chlorophenyl)-1-[(2,4-dimethoxyphenyl)sulphonyl]-2-oxo-2,3-dihydro-1H-indol-3-yl]-4-hydroxy-N,N-dimethyl-2-pyrrolidine-carboxamide;
methyl(2S,4R)-1-[5-chloro-1-[(2,4-dimethoxyphenyl) sulphonyl]-3-(2-methoxyphenyl)-2-oxo-2,3-dihydro-1H-indol-3-yl]-4-methoxy-2-pyrrolidinecarboxylate;
methyl(2S,4R)-1-[5-chloro-1-[(2,4-dimethoxyphenyl)sulphonyl]-3-(2-methoxyphenyl)-6-methyl-2-oxo-2,3-dihydro-1H-indol-3-yl]-4-methoxy-2-pyrrolidine-carboxylate;
(2S,4R)-1-[5-chloro-1-[(2,4-dimethoxyphenyl)sulphonyl]-3-(2-ethoxyphenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl)sulphonyl]-3-(2,3-difluorophenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl) sulphonyl]-3-(2,4-dimethoxyphenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl)sulphonyl]-3-(1,3-benzodioxol-4-yl)-2-oxo-2,3-dihydro-1H-indol-3-yl]-4-hydroxy-N,N-dimethyl-2-pyrrolidine-carboxamide;
(2S,4R)-1-[5,6-dichloro-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide;
tert-butyl 2-[[(3R,5S)-1-[5-chloro-1-[(2,4-dimethoxyphenyl) sulphonyl]-3-(2-methoxyphenyl)-2-oxo-2,3-dihydro-1H-indol-3-yl]-5-[(dimethylamino)carbonyl]-3-pyrrolidinyl]oxy]acetate;
2-[[(3R,5S)-1-[5-chloro-1-[(2,4-dimethoxyphenyl)sulphonyl]-3-(2-methoxyphenyl)-2-oxo-2,3-dihydro-1H-indol-3-yl]-5-[(dimethylamino)carbonyl]-3-pyrrolidinyl]oxy]acetic acid;
(2S,4R)-1-[5-chloro-1-[(2,4-dimethoxyphenyl)sulphonyl]-3-(2-methoxyphenyl)-2-oxo-2,3-dihydro-1H-indol-3-yl]-4-[2-[[2-hydroxy-1-(hydroxymethyl)-1-methylethyl]amino]-2-oxoethoxy]-N,N-dimethyl-2-pyrrolidinecarboxamide;
(2S,4R)-1-[5-chloro-1-[(2,4-dimethoxyphenyl)-sulphonyl]-3-(2-methoxyphenyl)-2-oxo-2,3-dihydro-1H-indol-3-yl]-N,N-dimethyl-4-[2-oxo-2-(1-piperazinyl)ethoxy]-2-pyrrolidinecarboxamide;
(2S,4R)-1-[[(2,4-dimethoxyphenyl)sulphonyl]-3-(2-methoxyphenyl)-2-oxo-2,3-dihydro-1H-indol-3-yl]-N,N-dimethyl-4-[2-oxo-2-(4-morpholinyl)ethoxy]-2-pyrrolidinecarboxamide;
(3R,5S)-1-[5-chloro-1-[(2,4-dimethoxyphenyl)sulphonyl]-3-(2-methoxyphenyl)-2-oxo-2,3-dihydro-1H-indol-3-yl]-5-[(dimethylamino)carbonyl]-3-pyrrolidinyl 3-(4-morpholinyl)propanoate;
   including the laevorotatory and dextrotatory isomers thereof, as well as the possible salts thereof with mineral or organic acids, and the solvates and/or hydrates thereof. These compounds may be synthesized as described, for example, in U.S. 2003/0114683, the disclosure of which is incorporated herein by reference in its entirety.

In yet another embodiment, the V_{1b} receptor antagonist shows competitive nanomolar affinity for animal and human V_{1b} receptors and lower affinity for animal and human V₁ₐ, V₂, and oxytocin receptors. In a related embodiment, the V_{1b} receptor antagonist behaves as a full antagonist in vitro and inhibits arginine vasopressin (AVP)-induced Ca²⁺ increase in Chinese hamster ovary cells expressing animal or human V_{1b} receptors.

In yet another embodiment, (2S,4R)-1-[5-Chloro-1-[(2,4-dimethoxyphenyl)-sulfonyl]-3-(2-methoxy-phenyl)-2-oxo-2,3-dihydro-1H-indol-3-yl]-4-hydroxy-N,N-dimethyl-2-pyrrolidine carboxamide (SSR149415) is able to restore normal thermoregulation in the OVX-induced telemetry model of thermoregulatory function.

In yet another embodiment, antagonists may be identified by selecting those substances which antagonize the biological activity of a V_{1b} agonist, such as AVP, at the AVP V_{1b} receptor in recombinant cell-lines or native cell-lines expressing AVP V_{1b} receptor, or cell cultures prepared from anterior pituitary (containing ACTH secreting cells or corticotrophs). Accordingly, the present invention provides a method for identifying agents for use in the treatment of vasomotor instability symptoms comprising: (a) growing a cell or tissue sample in the presence and absence of a test agent, wherein the cell or tissue expresses a V_{1b} receptor (endogenously expressed or over-expressed), (b) determining the biological activity of a V_{1b} receptor agonist (e.g. AVP) at the V_{1b} receptor through, (c) comparing said activity in the presence of the test agent to the activity in the absence of the test agent to identify an agent that antagonizes the activity of V_{1b} agonist, wherein reduced activity in the presence of the test agent identifies an agent that would alleviate vasomotor instability symptoms. The identified agent would have V_{1b} receptor antagonist activity. Various cell lines or tissues such as CHO-K1 cells expressing recombinant V_{1b} receptor or cell cultures prepared from anterior pituitary (containing ACTH secreting cells or corticotrophs) can be used (see PNAS 2002 99 6370-6375; J Pharmacol Exp Ther 2002 300: 1122-1130, and Endocrinology 2002143, No. 12 4655-4664).

Biological activity of V_{1b} agonists in cells expressing V_{1b} can be determined by various methods known in the art. In one embodiment, the ability to increase the concentration of intracellular calcium ([Ca⁺⁺]ᵢ) be measured (Am. J. Physiol. Endocrinol. Metab. 2003 285 E566-E576). Changes in [Ca²⁺] can be measured using a combination of calcium sensitive fluorescent indicator dyes (e.g. Fluo-3, Fluo-4 or Calcium Green 1) and automated fluorescent detection systems (e.g. Flex Station (Molecular Devices) or Fluorometric Imaging Plate Reader system (FLIPR®, Molecular Devices) platforms (Am. J. Physiol. Endocrinol. Metab. 2003 285 E566-E576).

In another embodiment, the increase in the hydrolysis of phosphatidyl inositol-4,5-bisphosphate (PIP₂) by phospholipase C into inositol-1,4,5-triphosphate (IP₃) and diacylglyceraldehyde (DAG) can be determined. In yet another embodiment, the generation of inositol 1,4,5-triphosphate (IP₃), or other intracellular signaling products of V_{1b} receptor coupling to Gq/11 can be measured.

An example of in vitro screen for identifying agents for use in the treatment of vasomotor instability involves: measuring or, detecting, quantitatively or qualitatively, the binding affinity of a test agent to the V_{1b} receptor (or to the cells or membranes expressing the V_{1b} receptor) by means of a label directly or indirectly associated with the test agent. The screen may further comprise determining the test agent's ability to displace binding of AVP to the V_{1b} receptor.

Cells used in these in vitro functional screens include but are not limited to native cell lines (e.g. corticotrophs), or cells expressing the V_{1b} receptor that have been isolated from native tissues, such as the anterior pituitary. Examples of cell-lines that may host recombinant expression of V1b may include but are not limited to Chinese Hamster Ovary (CHO), Human Embryonic Kidney 293 (HEK293), mouse fibroblast NIH3T3, and mouse LMTK-cells.

Cell membranes expressing the V_{1b} receptor from native tissues (e.g. anterior pituitary, or cell lines expressing recombinant AVP V_{1b} receptor) can be prepared according to methods known in the art. For instance, whole cells are harvested and the cell pellet is disrupted by sonication in ice cold buffer (e.g. 20 mM Tris HCl, mM EDTA, pH 7.4 at 4° C.). The resulting crude cell lysate is cleared of cell debris by low speed centrifugation at 200 times gravity (×g) for 5 minutes at 4° C. The cleared supernatant is then centrifuged at 40,000×g for 20 minutes at 4° C. and the resulting membrane pellet is washed by suspending in ice cold buffer and repeating the high speed centrifugation step. The final washed membrane pellet is re-suspended in assay buffer. Protein concentrations are determined by the method of Bradford (1976) using bovine serum albumin as a standard. The membranes may be used immediately or frozen for later use.

In yet other embodiments, the invention is directed to pharmaceutical compositions for treating at least one vasomotor symptom in a subject, comprising:
a. at least one V_{1b} receptor antagonist or a pharmaceutically acceptable salt thereof; and
b. at least one pharmaceutically acceptable carrier.

Generally, the V_{1b} receptor antagonist or a pharmaceutically acceptable salt thereof will be present at a level of from about 0.1%, by weight, to about 90% by weight, based on the total weight of the pharmaceutical composition. Preferably, the V_{1b} receptor antagonist or a pharmaceutically acceptable salt thereof will be present at a level of at least about 1%, by weight. More preferably, the V_{1b} receptor antagonist or a pharmaceutically acceptable salt thereof will be present at a level of at least about 5%, by weight. Even more preferably, the V_{1b} receptor antagonist or a pharmaceutically acceptable salt thereof will be present at a level of at least about 10%, by weight. Yet even more preferably, the V_{1b} b receptor antagonist or a pharmaceutically acceptable salt thereof will be present at a level of at least about 25%, by weight.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

The abbreviations in the specification correspond to units of measure, techniques, properties or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean, “IU” means International Units, “TST” means tail skin temperature, “sc” means subcutaneous, “OVX” means ovariectomized, and “DMSO” is dimethyl sulfoxide. “Δ° C.” and ΔTST mean change in tail skin temperature normalized for 15 minutes baseline TST prior to naloxone-induced flush. “ED₅₀ value” means dose which results in 50% alleviation of flush (50% mean maximum endpoint).

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

In the context of this disclosure, a number of terms shall be utilized. The following definitions are provided for the full understanding of terms and abbreviations used in this specification.

The phrases “vasomotor symptom,” “vasomotor instability”, “vasomotor dysfunction” and “vasomotor disturbance” include, but are not limited to, hot flushes (flashes), insomnia, sleep disturbances, mood disorders, irritability, excessive perspiration, night sweats, fatigue, and the like, caused by, inter alia, thermoregulatory dysfunction.

The term “hot flush” is an art-recognized term that refers to an episodic disturbance in body temperature typically consisting of a sudden skin flushing, usually accompanied by perspiration in a subject.

The terms “treatment” and “treating,” as used herein includes preventative (e.g., prophylactic), curative or palliative treatment and “treating” as used herein also includes preventative, curative and palliative treatment.

The term “effective amount” refers to an amount effective, at dosages and for periods of time necessary, to achieve the desired result. In particular, “effective amount” refers to the amount of compound or composition of compounds that would modulate V_{1b} activity for treating vasomotor symptoms in a subject in need thereof.

It will be appreciated that the effective amount of components of the present invention will vary from patient to patient not only with the particular compound, component or composition selected, the route of administration, and the ability of the components (alone or in combination with one or more combination drugs) to elicit a desired response in the individual, but also with factors such as the disease state or severity of the condition to be alleviated, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects.

Preferably, the compounds of the present invention, are administered at a dosage and for a time such that the number of hot flushes is reduced as compared to the number of hot flushes prior to the start of treatment. Such treatment can also be beneficial to reduce the overall severity or intensity distribution of any hot flushes still experienced, as compared to the severity of hot flushes prior to the start of the treatment.

For example, for a patient who experiences any number of hot flushes, compounds having vasopressin receptor antagonist activity can be administered preferably at a dosage of from 0.1 to 300, more preferably from 1 to 150 and most preferably from 1 to 50 mg/day for a time that the number and/or severity of hot flushes is reduced or such that hot flushes are substantially eliminated.

The terms “composition of compounds”, “compound”, “drug” or “pharmacologically active agent” or “agent” or “medicament” are used interchangeably herein to refer to a compound or compounds or composition of matter which, when administered to a subject (human or animal) induces a desired pharmacological and/or physiologic effect by local and/or systemic action. The component herein may contain vasopressin receptor antagonist activity. The term “test compound” or “test agent” refer to any compound or combination of more than one compound that is to be tested.

The term “modulation” refers to the capacity to either enhance or inhibit a functional property of a biological activity or process, for example, receptor binding, signaling activity. Such enhancement or inhibition may be contingent on the occurrence of a specific event, such as activation of a vasopressin receptor and/or may be manifest only in particular cell types. The modulator is intended to comprise any molecule, e.g., antibody, small molecule, peptide, antisense oligonucleotide, oligopeptide, polypeptide, or protein, preferably small molecule or peptide.

The term “inhibit” refers to the act of diminishing, suppressing, alleviating, preventing, reducing or eliminating, whether partial or whole, a function or an activity. The term “inhibit” can be applied to both in vitro as well as in vivo systems. As used herein, the term “inhibitor” refers to any agent that inhibits. The inhibitor may exhibit its partial, complete, competitive and/or inhibitory effect on mammalian, preferably the human vasopressin receptor, thus diminishing or blocking, preferably diminishing, some or all of the biological effects of endogenous vasopressin. Examples of vasopressin receptor inhibitors are: SR-49059 (V₁ₐ receptor) (1993 Serradeil-Le Gal, et al., J Clin Invest, 92(1)), OPC-31260 (V₂ receptor) (1992 Yamamura, et al., Br J Pharmacol, 105(4)), SSR-149415 (V_{1b} receptor) (2002 Serradeil-Le Gal, et al., JPET, 300(3)), YM-087 (both V₁ₐ and V₂ receptors) (1997 Tahara et al, JPET, 282(1)),

A suitable antagonist is one which binds to the receptor thereby denying access to that receptor by the natural ligand or otherwise prevents agonist activity. The antagonist may bind at or near the active site of the receptor. A “V_{1b} receptor antagonist” refers to a compound that interferes with or inhibits the binding of vasopressin to a V_{1b} receptor.

V_{1b} receptor modulators include, but are not limited, to non-peptide and peptide molecules.

“Non-peptide” refers to a compound which comprises preferably less than three amide bonds in the backbone core compound or preferably less than three amino acids or amino acid mimetics.

“Peptide” refers to an oligomer of at least two contiguous amino acid residues. Accordingly, the term “peptide” refers to any molecule comprising a string of amino acids, such as for example a peptide, a polypeptide, a protein or an antibody (or antibody fragment or derivative), which, if necessary, is modified or combined with other compounds or chemical groups.

Within the present invention, the V_{1b} receptor modulators may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

“Administering,” as used herein, means either directly administering a compound or composition of the present invention, or administering a prodrug, derivative or analog which will form an equivalent amount of the active compound or substance within the body.

The present invention includes prodrugs of V_{1b} receptor antagonists. “Prodrug,” as used herein, means a compound which is convertible in vivo by metabolic means (e.g. by hydrolysis) to a V_{1b} receptor antagonists. Various forms of prodrugs are known in the art, for example, as discussed in Bundgaard, (ed.), Design of Prodrugs, Elsevier (1985); Widder, et al. (ed.), Methods in Enzymology, Volume 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975).

Within the present invention, V_{1b} receptor antagonists may be prepared in the form of pharmaceutically acceptable salts, including salts of organic acids and minerals.

Further, the compounds of the present invention may exist in unsolvated as well as in solvated forms with pharmaceutically acceptable solvents such as water, ethanol, and the like. In general, the solvated forms are considered equivalent to the unsolvated forms for the purpose of the present invention.

A pharmaceutical composition for use in accordance with the present invention comprises a V_{1b} receptor antagonist, or a pharmaceutically acceptable salt thereof, together with a pharmaceutically acceptable carrier. The composition may comprise one or more V_{1b} receptor antagonist, as active ingredient(s), together with one or more pharmaceutically acceptable carrier(s).

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, N.Y., 1962); Wilen, S. H. Tables of Resolving Agents and Optical Resolutions, p. 268 (E. L. Eliel, Ed., University of Notre Dame Press, Notre Dame, Ind. 1972).

The term “laevorotatory isomer” refers to the optically active molecule that rotate polarized light to the left (counterclockwise). The “dextrorotatory isomer” refers to the optically active molecule that rotate the polarized light to the right (clockwise).

The term “combination therapy” refers to the administration of two or more agents or compounds to treat a therapeutic condition or disorder described in the present disclosure, for example hot flush, sweating, thermoregulatory-related condition or disorder, or other. Such administration includes co-administration of these agents or compounds in a simultaneous manner, such as in a single compound containing several V_{1b} receptor antagonists or in multiple, separate compounds. In addition, such administration also includes use of each type of agent in a concurrent manner. In either case, the treatment regimen will provide beneficial effects of the drug combination in treating the conditions or disorders described herein.

The route of administration may be any route, which effectively transports the V_{1b} receptor antagonists to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of one or more V_{1b} receptor antagonists may be concurrent or simultaneous.

The term “subject” refers to an animal, or to one or more cells derived from an animal. Preferably, the animal is a mammal, most preferably a human. Cells may be in any form, including but not limited to cells retained in tissue, cell clusters, immortalized cells, transfected or transformed cells, and cells derived from an animal that have been physically or phenotypically altered. The term “patient” refers to a subject which is an animal.

The term “patient” or “patients” is intended to refer to both the male and female gender unless one gender is specifically indicated. Accordingly, the term “patient” comprises any mammal which may benefit from treatment or prevention of vasomotor disturbances, such as a human, especially if the mammal is female, either in the pre-menopausal, peri-menopausal, or post-menopausal period. Furthermore, the term patient comprises female animals including humans and, among humans, not only women of advanced age who have passed through menopause but also women who have undergone hysterectomy or for some other reason have suppressed estrogen production, such as those who have undergone long-term administration of corticosteroids, suffer from Cushing's syndrome or have gonadal dysgenesis.

The terms “premature menopause” or “artificial menopause” refer to ovarian failure of unknown cause that may occur before age 40. It may be associated with smoking, living at high altitude, or poor nutritional status. Artificial menopause may result from oophorectomy, chemotherapy, radiation of the pelvis, or any process that impairs ovarian blood supply.

The term “pre-menopausal” means before the menopause, the term “peri-menopausal” means during the menopause and the term “post-menopausal” means after the menopause. “Ovariectomy” means removal of an ovary or ovaries and can be effected according to Merchenthaler et al., Maturitas, 1998, 30(3): 307-316.

The term “V_{1b} receptor” may be used interchangeably with the terms AVP V_{1b} receptor, vasopressin V_{1b} receptor, V1bR, V3, V3R, VPR3, AVPR V_{1b}, AVPR V3, and antidiuretic hormone receptor 1 b.

The term “V_{1b} receptor” intended to include all V_{1b} receptor variants. The term “Variant” refers to a V_{1b} receptor polypeptide which is a member of a family of polypeptides that are encoded by a single gene or from a gene sequence within a family of related genes and which may differ in their pl or MW, or both. Such variants can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation). The term variant further includes V_{1b} receptor “fragments”, “analogs”, and “derivatives” or any combination thereof.

The term “fragment”, “analog”, and “derivative” when referring to the V_{1b} receptor refers to a V_{1b} receptor polypeptide which retains essentially the same biological function or activity as V_{1b} receptor. For the purpose of the present invention, variants of the aforementioned polypeptides, includes all allelic forms and splice variants.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion, embodiments and these examples, one skilled in the art will readily appreciate that many modifications are possible in the exemplary embodiments without materially departing from the novel teachings of this inventions, and without departing from the spirit and scope thereof. Furthermore, one can make various changes to and modifications of the invention to adapt it to various usages and conditions. It is, therefore, intended that the appended claims cover all such equivalent variations as fall within the true spirit and scope of the invention.

General Methods

Reagents: The V_{1b} receptor antagonist SSR149415 (prepared as described in WO 01/55134 A2), V₁ₐ receptor antagonist SR49059 (prepared as described in U.S. 20030008860) and V₂ receptor antagonist VPA-985 (prepared as described in U.S. Pat. No. 5,516,774). The following reagents were purchased commercially: Morphine alkaloid pellets (Murty Pharmaceuticals, Lexington, Ky.), ketamine (Phoenix Pharmaceuticals, Belmont, Calif.), and naloxone (Research Biochemicals International, St. Louis, Mo.).

Dosing: All doses were prepared based on mg/kg. All compounds were administered intraperitoneally and dissolved and used at the following dosages: SSR149415 (V_{1b} receptor antagonist, 3.0-30 mg/kg was dissolved in 5% DMSO in 5% Cremophor EL). SSR49059 (V₁ₐ receptor antagonist, 30 mg/kg was dissolved 10% DMSO/sterile H₂O) and VPA-985 (V₂ receptor antagonist, 30 mg/kg was dissolved 10% DMSO/sterile H₂O). Naloxone (1.0 mg/kg was dissolved in sterile H₂O) was injected subcutaneously. Ketamine (Ketaject, Phoenix Pharmaceuticals, Belmont, Calif.) was injected intramuscularly at a dosage (40 mg/kg) that was determined to be mildly sedative but did not cause a change in tail skin temperature.

Animals: Ovariectomized Sprague-Dawley rats (180-220 g) were obtained from a commercial vendor (Taconic, Germantown, N.Y.) and individually housed under 12 hours light/dark cycle in a room maintained at 25° C. Animals were provided with standard rat chow and water ad libitum.

Morphine-dependent model: Ovariectomized rats were injected once daily for 8-9 days with vehicle to minimize stress responses and then administered compound(s) on test day (10 rats/group). On day 4 of vehicle dosing, morphine dependence was induced by sc implantation of two slow-release morphine pellets (75 mg/pellet) in the dorsal scapular region. This model is based upon an established morphine-dependent naloxone-induced flush paradigm that is reversible by estrogen treatment (Katovich et al., Proceedings of the Society for Experimental Biology & Medicine, 1990. 193(2): p. 129-35). Four to six days after implantation, morphine withdrawal was induced with an opioid antagonist (naloxone) that causes a transient increase in TST. In a typical experiment, rats were administered their final dose of test compound 40 minutes prior to naloxone injection. Rats were mildly sedated with ketamine and a thermistor connected to a MacLab data acquisition system (CB Sciences, Dover, N.H.) was taped to the base of the tail. Tail skin temperature was then monitored continuously for 10 minutes to establish a baseline temperature. Naloxone was subsequently administered and TST was measured for an additional 35 minutes (total recording time 95 min).

OVX-induced thermoregulatory dysfunction telemetry model: To determine the effect that test compounds had on thermoregulation, TST was monitored in ovariectomized rats by telemetry (6-7 rats/group). This model has been modified from a previously reported protocol based on estrogen regulation of diurnal TST patterns (Berendsen et al, European Journal of Pharmacology, 2001. 419(1): p. 47-54). Following acclimation, a temperature and physical activity transmitter (PhysioTel TA10TA-F40, Data Sciences International) was implanted subcutaneously in the dorsal scapular region and the tip of temperature probe was tunneled subcutaneously 2.5 cm beyond the base of the tail. After a 7 day recovery period rats were administered a vehicle injection and TST monitored continuously for 12 hours (Day 1). Twenty-four hours later rats were administered either vehicle or test compound and TST monitored continuously for 12 hours (Day 2). Since TST varies between the active (dark) and inactive (light) phase over a 24 hours period, effects of test compounds were evaluated during the dark cycle. All vehicle and test compounds were administered 30 minutes prior to the onset of the dark cycle.

Statistical analysis: To analyze changes in TST induced by naloxone in morphine-dependent rats, all data was analyzed using a two factors repeated measure. The factors were “treatment” and “time” (repeated). The model was fit to test whether there were significant differences in the responses between treatment groups. The data was analyzed at 5 minute intervals from 20 minutes (−20) prior to the naloxone administration (referred to as time 0) to 60 minutes after the treatment. Multiple comparisons (LSD p-values) among the treatment groups at each time point were used for the analysis, however, the changes in TST is greatest at 15 minutes post naloxone administration and this time point provides the best indicator of flush abatement. When appropriate estimation of the ED₅₀ value was calculated. The ED₅₀ value was determined using a log scale and the line was fit between the maximal (15 minutes post naloxone ΔTST) and minimal response (average baseline temperature prior to naloxone). The ED₅₀ value is reported as the dose of test compound that abates 50% of the naloxone-induced flush.

To analyze changes in TST induced by test compounds in the thermoregulatory dysfunction model a 2-day paradigm was used. For both vehicle (baseline) and compound testing, TST is recorded at 5 minute intervals and an average TST is calculated for every 30 minute time point. On Day 1, an overall average baseline TST is established for each animal by taking the mean over the 12 hours observation period. On Day 2, test compound is administered and TST readings are recorded every 30 minutes as described above. All data were analyzed as ΔTST (TST for each time point Day 2−average baseline TST Day 1). A one-way ANOVA is performed to obtain the average within-group standard deviation for that compound. For each 30 minute interval, a t-test is performed and evaluated to determine if the average ΔTST is statistically different (p<0.05) from zero. Data will be presented as mean ΔTST, duration of effect during active phase and overall activity index (Mean ΔTST×Duration).

### EXAMPLE 1

### Effect of V_{1b} Receptor Antagonist SSR149415 in Alleviating Vasomotor Instability in Pre-Clinical Models of Vasomotor Instability

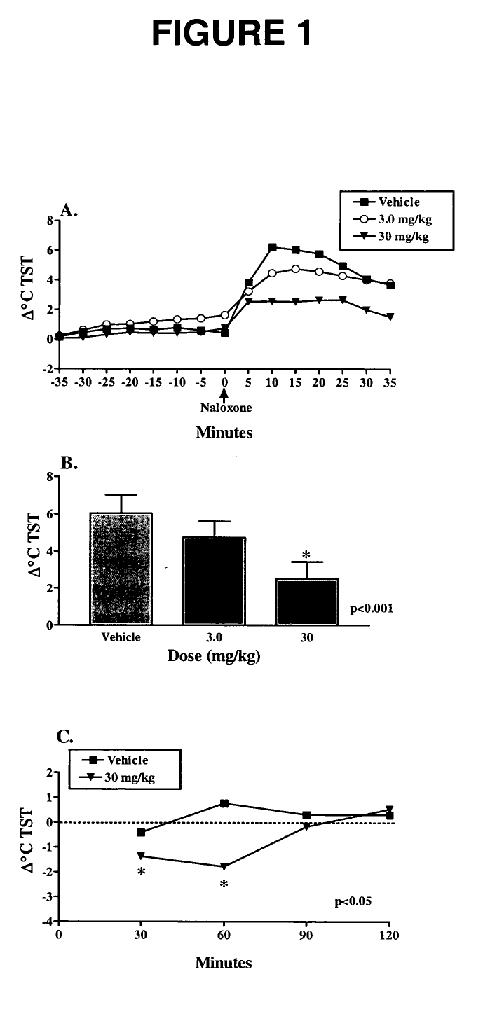

Method used as described in the general method section under morphine-dependent rat model with the following exceptions: in the morphine-dependent model rats were injected intraperitoneally with vehicle (5% DMSO in 5% Cremophor EL) or SSR149415 (V_{1b} receptor antagonist) which may be prepared as described in WO 01/55134, dissolved in 5% DMSO in 5% Cremophor EL and administered at 3.0 and 30 mg/kg 40 minutes prior to naloxone injection (FIGS. 1A and 1B). Changes in TST (Δ° C., Mean) over time in the morphine-dependent rat model demonstrate that the V_{1b} receptor antagonist SSR149415 dose-dependently abates the naloxone-induced flush (FIG. 1A). At maximal flush (15 minutes post-naloxone; Δ° C., Mean+SEM) SSR149415 dose-dependently (ED₅₀ value=12.1+8 mg/kg) abates the naloxone-induced flush (FIG. 1B). At the highest dose (30 mg/kg) tested antagonism of the V_{1b} receptor produced a 60% abatement of the naloxone-induced flush.

In the OVX-induced thermodysregulation model rats were injected intraperitoneally with vehicle (sterile H₂O or 5% DMSO in 5% Cremophor EL) or SSR149415 dissolved in 5% DMSO in 5% Cremophor EL and administered at 30 mg/kg (FIG. 1C). Changes in TST (Δ° C., Mean+SEM) over time in the telemetry model of OVX-induced thermodysregulation demonstrate that SSR149415 significantly and transiently decreased TST during the active phase (FIG. 1C).

Antagonism of the V_{1b} receptor was efficacious in a naloxone-induced flush rat model of vasomotor instability and was able to transiently restore normal thermoregulation in the OVX-induced telemetry model of thermoregulatory dysfunction.

### EXAMPLE 2

### Lack of Effect of V₁ₐ and V₂ Receptor Antagonists in Alleviating Vasomotor Instability

Method are described in the general method section under morphine-dependent rat model with the following exceptions: Rats were injected intraperitoneally with vehicle (10% DMSO/sterile H₂O), V₁ₐ receptor antagonist SR49059 (may be prepared as described in U.S. 2003008860, dissolved in 10% DMSO/sterile H₂O at 30 mg/kg) or V₂ receptor antagonist VPA-985 (may be prepared as described in U.S. Pat. No. 5,516,774, dissolved in 10% DMSO/sterile H₂O at 30 mg/kg) administered 40 minutes prior to naloxone injection.

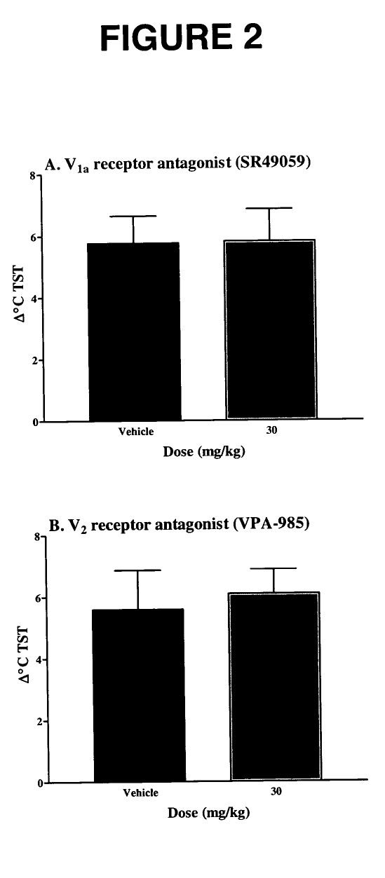

At maximal hot flush (15 minutes post-naloxone; Δ° C., Mean+SEM) neither the V₁ₐ receptor antagonist SR49059 nor the V₂ receptor antagonist VPA-985 significantly abated the naloxone-induced flush (FIGS. 2A and 2B). In contrast, to the V_{1b} receptor antagonist both V₁ₐ and V₂ receptor antagonists lack efficacy in attenuating the naloxone-induced flush and suggest a selectivity of effect for V_{1b} receptor antagonism and alleviation of vasomotor instability.

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method for treating at least one vasomotor symptom in a subject in need thereof, comprising the step of:
administering to said subject an effect amount of a composition, comprising:
at least one compound that modulate the biological activity of V_{1b} receptor or pharmaceutically acceptable salt thereof.

2. A method according to claim 1,
wherein said compound is V_{1b} receptor antagonist.

3. A method according to claim 2,
wherein said V_{1b} receptor antagonist is a non-peptide or a peptide molecule.

4. A method according to claim 1,
wherein said compound is:
(2S,4R)-1-[5-chloro-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl) sulphonyl]-3-(2-methoxyphenyl)-2-oxo-2,3-dihydro-1H-indol-3-yl]-4-methoxy-N,N-dimethyl-2-pyrrolidine-carboxamide;
(2S,4R)-1-[5-chloro-3-(2-chlorophenyl)-1-[(2,4-dimethoxyphenyl)sulphonyl]-2-oxo-2,3-dihydro-1H-indol-3-yl]-4-hydroxy-N,N-dimethyl-2-pyrrolidinecarboxamide;
(2S,4R)-1-[5-chloro-3-(2-chlorophenyl)-1-[(2,4-dimethoxyphenyl)sulphonyl]-6-methoxy-2-oxo-2,3-dihydro-1H-indol-3-yl]-4-methoxy-N,N-dimethyl-2-pyrrolidine-carboxamide;
(2S,4R)-1-[5-chloro-1-[(3,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide;
methyl(2S,4R)-1-[5-chloro-3-(2-methoxyphenyl)-1-[(3,4-dimethoxyphenyl)sulphonyl]-2-oxo-2,3-dihydro-1H-indol-3-yl]-4-hydroxy-2-pyrrolidinecarboxylate;
(2S,4R)-1-[5-methyl-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl)-sulphonyl]-3-(2-methoxyphenyl)-2-oxo-2,3-dihydro-1H-indol-3-yl]-2-(azetidin-1-ylcarbonyl)-4-hydroxy-pyrrolidinecarboxamide;
(2S,4R)-1-[5-trifluoromethoxy-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidinecarboxamide;
(2S,4R)-1-[5-chloro-1-[(2,4-dimethoxyphenyl)sulphonyl]-3-(2-methoxyphenyl)-6-methyl-2-oxo-2,3-dihydro-1H-indol-3-yl]-4-hydroxy-N,N-dimethyl-2-pyrrolidinecarboxamide;
(2S,4R)-1-[3-(2-chlorophenyl)-1-[(2,4-dimethoxyphenyl)sulphonyl]-5,6-dimethyl-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl) sulphonyl]-3-(2,3-dimethoxyphenyl)-2-oxo-2,3-dihydro-1H-indol-3-yl]-4-methoxy-N,N-dimethyl-2-pyrrolidine-carboxamide;
(2S,4R)-1-[5-chloro-1-[(2,4-dimethoxyphenyl)sulphonyl]-3-(2-methoxyphenyl)-6-trifluoromethyl-2-oxo-2,3-dihydro-1H-indol-3-yl]-4-methoxy-N,N-dimethyl-2-pyrrolidinecarboxamide;
(2S,4R)-1-[6-chloro-1-[(2,4-dimethoxyphenyl)sulphonyl]-3-(2-methoxyphenyl)-5-methyl-2-oxo-2,3-dihydro-1H-indol-3-yl]-4-methoxy-N,N-dimethyl-2-pyrrolidinecarboxamide;
(2S,4R)-1-[5-chloro-1-[(2,4-dimethoxyphenyl) sulphonyl]-3-(2-methoxyphenyl)-2-oxo-2,3-dihydro-1H-indol-3-yl]-4-ethoxy-N,N-dimethyl-2-pyrrolidine-carboxamide;
(2S,4R)-1-[5-chloro-1-[(2,4-dimethoxyphenyl)sulphonyl]-3-(2,3-dimethoxyphenyl)-2-oxo-2,3-dihydro-1H-indol-3-yl]-4-hydroxy-N,N-dimethyl-2-pyrrolidine-carboxamide;
(2S,4R)-1-[5,6-dichloro-3-(2-chlorophenyl)-1-[(2,4-dimethoxyphenyl)sulphonyl]-2-oxo-2,3-dihydro-1H-indol-3-yl]-4-hydroxy-N,N-dimethyl-2-pyrrolidine-carboxamide;
methyl(2S,4R)-1-[5-chloro-1-[(2,4-dimethoxyphenyl) sulphonyl]-3-(2-methoxyphenyl)-2-oxo-2,3-dihydro-1H-indol-3-yl]-4-methoxy-2-pyrrolidinecarboxylate;
methyl(2S,4R)-1-[5-chloro-1-[(2,4-dimethoxyphenyl)sulphonyl]-3-(2-methoxyphenyl)-6-methyl-2-oxo-2,3-dihydro-1H-indol-3-yl]-4-methoxy-2-pyrrolidine-carboxylate;
(2S,4R)-1-[5-chloro-1-[(2,4-dimethoxyphenyl)sulphonyl]-3-(2-ethoxyphenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl)sulphonyl]-3-(2,3-difluorophenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl) sulphonyl]-3-(2,4-dimethoxyphenyl)-2-oxo-2,3-dihydro-1H-indol-3-yl]-4-hydroxy-N,N-dimethyl-2-pyrrolidine-carboxamide;
(2S,4R)-1-[5-chloro-1-[(2,4-dimethoxyphenyl)sulphonyl]-3-(1,3-benzodioxol-4-yl)-2-oxo-2,3-dihydro-1H-indol-3-yl]-4-hydroxy-N,N-dimethyl-2-pyrrolidine-carboxamide;
(2S,4R)-1-[5,6-dichloro-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide;
tert-butyl 2-[[(3R,5S)-1-[5-chloro-1-[(2,4-dimethoxyphenyl) sulphonyl]-3-(2-methoxyphenyl)-2-oxo-2,3-dihydro-1H-indol-3-yl]-5-[(dimethylamino)carbonyl]-3-pyrrolidinyl]oxy]acetate;
2-[[(3R,5S)-1-[5-chloro-1-[(2,4-dimethoxyphenyl)sulphonyl]-3-(2-methoxyphenyl)-2-oxo-2,3-dihydro-1H-indol-3-yl]-5-[(dimethylamino) carbonyl]-3-pyrrolidinyl]oxy]acetic acid;
(2S,4R)-1-[5-chloro-1-[(2,4-dimethoxyphenyl)sulphonyl]-3-(2-methoxyphenyl)-2-oxo-2,3-dihydro-1H-indol-3-yl]4-[2-[[2-hydroxy-1-(hydroxymethyl)-1-methylethyl]amino]-2-oxoethoxy]-N,N-dimethyl-2-pyrrolidinecarboxamide;
(2S,4R)-1-[5-chloro-1-[(2,4-dimethoxyphenyl)-sulphonyl]-3-(2-methoxyphenyl)-2-oxo-2,3-dihydro-1H-indol-3-yl]-N,N-dimethyl-4-[2-oxo-2-(1-piperazinyl)ethoxy]-2-pyrrolidinecarboxamide;
(2S,4R)-1-[[(2,4-dimethoxyphenyl)sulphonyl]-3-(2-methoxyphenyl)-2-oxo-2,3-dihydro-1H-indol-3-yl]-N,N-dimethyl-4-[2-oxo-2-(4-morpholinyl)ethoxy]-2-pyrrolidinecarboxamide;
(3R,5S)-1-[5-chloro-1-[(2,4-dimethoxyphenyl)sulphonyl]-3-(2-methoxyphenyl)-2-oxo-2,3-dihydro-1H-indol-3-yl]-5-[(dimethylamino)carbonyl]-3-pyrrolidinyl 3-(4-morpholinyl)propanoate;
or a pharmaceutically salt thereof.

5. A method according to claim 4,
wherein said compound is a laevorotatory isomer.

6. A method according to claim 1,
wherein said V_{1b} receptor antagonist is (2S,4R)-1-[5-chloro-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide.

7. A method according to claim 6,
wherein said (2S,4R)-1-[5-chloro-1-[(2,4-dimethoxyphenyl)sulphonyl]-3-(2-methoxyphenyl)-2-oxo-2,3-dihydro-1H-indol-3-yl]-4-hydroxy-N,N-dimethyl-2-pyrrolidine-carboxamide is a laevorotatory isomer.

8. A method according to claim 1,
wherein said vasomotor symptom is hot flushes, insomnia, sleep disturbances, mood disorders, irritability, excessive perspiration, night sweats, fatigue, or a combination thereof.

9. A method according to claim 1,
wherein said subject is human.

10. A method according to claim 9,
wherein said human is a female.

11. A method according to claim 10,
wherein said female is pre-menopausal.

12. A method according to claim 10,
wherein said female is peri-menopausal.

13. A method according to claim 10,
wherein said female is post-menopausal.

14. A method according to claim 9,
wherein said human is a male.

15. A method according to claim 14,
wherein said male is naturally, chemically or surgically andropausal.

16. A method for identifying an agent for treating at least one vasomotor symptom in a subject, comprising the steps of:
growing a cell or tissue sample in the presence and absence of a test agent, wherein said cell or tissue sample expresses a V_{1b} receptor;
determining the biological activity of a V_{1b} agonist at said V_{1b} receptor in the presence and the absence of said agent; and
identifying said agent that antagonizes or reduces said biological activity of said V_{1b} agonist.

17. A method according to claim 16,
wherein said V_{1b} receptor is endogenously expressed.

18. A method according to claim 16,
wherein said V_{1b} receptor is over-expressed.

19. A method for screening to identify an agent for treating at least one vasomotor symptom in a subject, comprising the step of:
determining a binding affinity of said agent to a V_{1b} receptor.

20. A method according to claim 19, further comprising the step of:
determining the ability of said agent to displace binding of vasopressin to said V_{1b} receptor.

21. A method for screening to identify an agent for treating at least one vasomotor symptom in a subject, comprising the step of:
determining a binding affinity of said agent to cells or membranes expressing a V_{1b} receptor.

22. A pharmaceutical composition for treating at least one vasomotor symptom in a subject, comprising:
a. at least one V_{1b} receptor antagonist or a pharmaceutically acceptable salt thereof; and
b. at least one pharmaceutically acceptable carrier.
